(19) United States
(12) Patent Application Publication
(10) Pub. No.: US 2013/0165957 A1
(43) Pub. Date: Jun. 27, 2013

(54) Implantable Prosthetic Devices and Solvent-Casting Methods for Manufacturing Same

(71) Applicants: Gianluca Ciardelli, Pisa (IT); Chiara Tonda Turo, Carignano (Torino) (IT); Piergiorgio Gentile, Torino (IT); Valeria Chiono, Bonassola (La Spezia) (IT)

(72) Inventors: Gianluca Ciardelli, Pisa (IT); Chiara Tonda Turo, Carignano (Torino) (IT); Piergiorgio Gentile, Torino (IT); Valeria Chiono, Bonassola (La Spezia) (IT)

(73) Assignee: Politecnico Di Torino, Torino (IT)

(21) Appl. No.: 13/818,326

(22) PCT Filed: Aug. 30, 2011
(86) PCT No.: PCT/IB11/53787

§ 371 Date: Feb. 22, 2013

(30) Foreign Application Priority Data
Aug. 31, 2010 (IT) TO2010A000726

(51) Int. Cl. A61F 2/00

(52) U.S. Cl. CPC A61F 2/0063
USPC 606/151; 427/2.24

(57) ABSTRACT

Implantable prosthetic devices are provided. Such devices include substantially planar supports such as a mesh for surgical use made of polypropylene, and a porous coating membrane formed on at least one face of said support, said porous membrane being formed in situ on the support by the solvent casting technique. Methods for making such prosthetic devices are also provided.

The present invention relates to an implantable prosthetic device made of an absorbable or non-absorbable polymeric material and the method of manufacture thereof.

In particular, the invention relates to a prosthetic device comprising a substantially planar support of polymeric material, for example a mesh-like support of varying weight, mass, mesh size and thickness, of woven or non-woven type, as well as prosthetic devices having membrane or porous or non-porous film supports.

The prosthetic device to which the invention relates can for example be intended for applications in soft-tissue surgery.

The prosthetic device according to the invention preferably consists of a double layer characterized by a woven mesh for surgical use and a porous membrane (coating), for use as a prosthesis for hernia repair. This mesh can also be used as a prosthesis for the repair and reinforcement of the abdominal wall and of the inguinal region of the pelvic floor, for the treatment of incontinence or for the healing of burns.

Synthetic prostheses have been in regular use in surgery for decades. Among the commonest are monofilament meshes of polypropylene, which is a non-absorbable material. These devices do not reach the gold standard, owing to the intense foreign-body reaction that they induce and the rapid and massive colonization of fibroblasts to which they are subject. The complications resulting therefrom are primarily shrinkage of the mesh itself, chronic pain and the formation of a thick scar plate, which restricts the patient's movements and reduces the elasticity of the abdominal wall. Moreover, the strong adhesion with the tissues that these devices cause in the intestinal loops makes them unsuitable for intraperitoneal use. Some statistics show that relapses within 5 years can reach 40-50%. Abdominal hernia, i.e. protrusion of viscera through a defect of the aponeurotic muscle wall, represents one of the commonest complications of abdominal surgery. This complication arises, in elective procedures, with a frequency that reaches 8-10%. In emergency surgery, with abdominal septic phenomena or contamination of the surgical field, this value can go above 40%. Clinical relief, with all symptoms correlated, occurs in 65-70% of cases within the space of a year after the primary intervention. The treatment of relapses, which in the past was burdened with an intolerable failure rate (>55%), now envisages the routine use of prosthetic materials with a reduction of these values, in some case records, to less than 5%.

Therefore the main objective of scientific and industrial research has been to make materials available that are able to reduce the reaction of adhesion to the intestinal loops and at the same time are able to guarantee colonization by fibroblasts, to permit immobilization of the prosthesis in the abdominal wall. This has led to solutions such as composite meshes having an absorbable component, or double-layer meshes, the inner layer of which is made of a non-stick material. However, the latter solution means increased thickness of the prosthesis, which ultimately proves troublesome for the patient.

Among the absorbable materials most used for manufacturing prosthetic devices for pelvic and abdominal surgery, there is a membrane composed of hyaluronic acid and carboxymethyl cellulose (Seprafilm) which tends to absorb the moisture present in the implantation site, being transformed into a gel about 24 hours after application. In addition to reduction of tissue adhesions, Seprafilm membranes offer the advantage of being degraded in a period of about seven days from the time of implantation. However, application of this coating proves particularly difficult since its hydrophilic nature leads to adhesion thereof if there is any moisture, including that present on the surgeon's gloves with which it comes in contact during positioning in the abdominal cavity.

As alternatives to Seprafilm, the use of bioactive coatings containing heparin or taurolidine has been evaluated. Even though heparin prevents the production of fibrin, a fundamental step in the process of formation of adhesions, the anticoagulant properties of this agent and the consequent risks of haemorrhage mean that it is more difficult to use for applications in prosthetics. Taurolidine is a derivative of the amino acid taurine and is an antimicrobial agent with anticancer properties. However, recent research has demonstrated that the action of taurolidine within the intraperitoneal cavity is not selective, thus leading to the death of healthy cells in addition to the tumour cells.

Analysis of the activity of various biomaterials at the interface with the visceral peritoneum has shown that the structure, and in particular the porosity, of the biomaterial play a key role in the reduction of adhesions with the intestinal loops, in the rapid integration of the host tissue and in reduction of the formation of seromas. Patent application TO2007A000846 describes a composite prosthetic device that comprises a support of polymeric material, preferably of the mesh type, coated with a network of fibres and nanofibres of polymeric material. The coating of fibres and nanofibres is produced and laid down on the implantable polymeric support by the electrospinning technique, which utilizes interactions of an electrostatic nature to exert tensile forces. The prosthetic device described in TO2007A000846 does not solve the problem of the delamination effects associated with implantable devices in which the polymeric coating layer is laid down on the support of the device during manufacture thereof and so is subject to detachment.

The purpose of the present invention is therefore to provide a prosthetic device, in particular with a double layer formed from a mesh and a porous membrane (coating), which makes it possible to reduce the complications caused by the formation of adhesions with the tissues of the implantation site, such as fistulas or seromas, but which at the same time prevents the delamination effects with consequent detachment of the coating layer of the device.

A further purpose of the invention is to provide a prosthetic device, in particular with a double layer formed from a mesh and a porous membrane (coating), that is able to stimulate cellular colonization and tissue regeneration, limiting inflammatory and fibrotic processes.

These and other purposes are achieved by means of a prosthetic device and a method of manufacture thereof as defined in claim 10 and in claim 1 respectively.

The appended claims form an integral part of the technical teaching of the present description.

The polymeric materials suitable for use for making the porous membrane for coating the device of the invention are materials that are able to reduce and/or prevent the formation of adhesions and the erosion of tissues; to reduce adhesion and bacterial proliferation and/or stimulate cellular growth of a particular tissue. Among them, we may mention as examples polycaprolactone (PCL), polylactic acid (PLA), polyglycolic acid (PGA), polyurethane (PU) and copolymers thereof.

Among the pore-forming agents suitable for use for making the porous membrane of the coating of the device of the invention, we may mention as examples polyethylene oxide (PEO), polyvinylpyrrolidone (PVP), polyvinyl alcohol (PVA), glucose, sodium chloride (NaCl).

In a preferred embodiment, the porous membrane of the coating is made using polycaprolactone (PCL) as principal material and polyethylene oxide (PEO) as pore-forming agent, in any proportions. The weight ratio of PCL to PEO is preferably within the range 90/10 to 50/50, more preferably it is 90/10, 80/20, 70/30, 60/40 or 50/50. For preparing the polymer solutions, organic solvents are used, such as chloroform, methylene chloride or dimethylformamide.

In a particularly preferred embodiment, the solution of polymeric material used for making the porous membrane additionally comprises vitamin E, preferably at a concentration between 5 and 50 wt.% based on the total weight of the solution, more preferably at a concentration of 25 wt.% based on the total weight of the solution. Vitamin E is a known antioxidant that contributes to the maintenance of cellular integrity. Owing to the properties exerted by this factor following release from the porous membrane of the coating, it is possible to obtain the important benefit of reducing the inflammatory, coagulative and fibrotic processes that affect the tissues that interact with the prosthetic device and which, taken together, contribute to the onset of adhesions. At the same time, the action of vitamin E stimulates the processes of tissue regeneration, promoting integration of the device at the implantation site.

The coating membrane that characterizes the prosthetic device of the invention is obtained by the solvent casting process, which is based on the use of a pore-forming agent dispersed in the form of particles in a solution comprising a polymeric material dissolved in a first solvent. The solution is placed on at least one face of the substantially planar support of the prosthetic device. After evaporation of the first solvent and consequent formation of a membrane of polymeric material, the device is dipped in a second solvent that is able to dissolve the particles of pore-forming agent, thus resulting in the formation of pores in the membrane. The use of suitable proportions between the polymer of the solution and the pore-forming agent used makes it possible to produce a membrane with micropores of uniform morphology and homogeneous distribution. Therefore with this technique it is possible to manufacture prosthetic devices that offer the advantage of a coating membrane with porosity characteristics that are optimized for the passage of fluids into the implantation zone. This guarantees the diffusion of nutrients, the removal of metabolic wastes and at the same time prevents phenomena of fluid accumulation, which is the cause of seromas and haematomas.

In addition, the use of this process is particularly advantageous as it makes it possible to obtain formation of the porous coating membrane in situ, directly on the substantially planar support of the prosthetic device according to the invention. This reduces the risk of detachment of the porous coating membrane from the underlying support, avoiding the problems and the dangers that result from the delamination effects.

The following examples are supplied for purposes of illustration and do not limit the scope of the invention as defined in the appended claims.

### EXAMPLES

### 1. Preparation of Porous Polymer Membranes

Polymer membranes based on PCL/PEO blends were produced by the solvent casting technique. This envisages the operations of:
preparing the polymer solution (polymer/solvent) using magnetic stirring;
pouring the solution into a glass mould;
allowing the solvent to evaporate.

Solutions of mixtures based on PCL and PEO were prepared with varying weight ratio (%w/w) of the two polymers (see Table 1).

TABLE 1

PCL/PEO (% w/w)

| | 100/0 | 90/10 | 80/20 | 70/30 | 60/40 | 50/50 |
|---|---|---|---|---|---|---|
| PCL (g) | 0.50 | 0.45 | 0.40 | 0.35 | 0.30 | 0.25 |
| PEO (g) | 0 | 0.05 | 0.10 | 0.15 | 0.20 | 0.25 |

In both cases, a solution was prepared in chloroform (Fluka) at a concentration of 5% (w/v).

For each mixture, in particular, with the concentration of solution (5%) and the total volume of solvent (10 ml) fixed, the relative percentage of the two components in the mixture to be prepared was varied.

The system consisting of the polymers and the solvent was stirred by magnetic stirring under a fume hood to facilitate dissolution.

For each mixture, complete dissolution of the components in chloroform was obtained after about 1 hour. When the solution had become homogeneous, it was poured into glass Petri dishes with a diameter of about 6 cm.

The dishes were put under the hood for 24 hours, permitting evaporation of the solvent (CHCl₃) and obtaining the casting membrane.

After drying, the membranes based on PCL/PEO were weighed on an electronic balance. To remove the PEO (pore-forming agent) present in the membranes, the samples were immersed in doubly-distilled water and kept in an incubator for 24 hours at a constant temperature of 37° C. The membranes were taken out and rinsed in water and then dried again.

To evaluate the effective weight loss of the pore-forming agent, the sample membranes were weighed and the percentage by weight of pore-forming agent dissolved was calculated as follows:

$$\% pore \text{-} forming agent = \frac{P before immersion in water - P after immersion in water}{P before immersion in water} \times 100$$

After removal of the pore-forming agent the samples are designated as PCL 100i, PCL 90i, PCL 80i, PCL 70i, PCL 60i, PCL 50i.

### 2. Preparation of Porous Polymer Membranes Containing Vitamin E

Porous membranes containing vitamin E were prepared by solvent casting, in particular:
PCL/PEO 80/20+Vitamin E (25%)
PCL/PEO 70/30+Vitamin E (25%)
PCL/PEO 60/40+Vitamin E (25%)

The concentration of solution of PCL/PEO polymer blend (5%) and the total volume of solution (10 ml) were fixed for each mixture (Table 2). The solution was prepared in chloroform (Fluka) and the procedure was repeated twice to obtain two membranes for each type in order to guarantee repeatability of the tests.

TABLE 2

| | PCL/PEO | | |
|---|---|---|---|
| | 80/20 | 70/30 | 60/40 |
| PCL (g) | 0.4 | 0.35 | 0.3 |
| PEO (g) | 0.1 | 0.15 | 0.2 |
| Vit. E (μl) | 263 | 263 | 263 |

All the membranes were immersed in doubly-distilled water to promote loss of PEO and were kept in an incubator at 37° C., then they were rinsed in water and dried again.

After the removal of pore-forming agent, the samples were designated as PCL 80i+vitE, PCL 70i+vitE, PCL 60i+vitE.

### 3. Morphological Analysis (SEM) of Porous Membranes of PCL Containing Vitamin E

Micrographs (surfaces and sections) of porous membranes based on PCL manufactured using the pore-forming agent PEO, in increasing percentages by weight, were analysed to evaluate the degree of dispersion of the pore-forming material in the PCL matrix and the porosity thus obtained.

Prior to removal of the PEO, the sections of the membranes were found to be denser and more compact, with a granular morphology typical of the polymer blends; however, pores were found to be present with a size of about 1 μm, due to the inclusion of air bubbles during mixing and/or to the incorporation of traces of water on account of the hygroscopicity of PEO, i.e. its capacity to absorb atmospheric moisture.

Following removal of the PEO, the porosity of the membranes was found to have increased: the density of pores and their size had increased relative to the corresponding membranes still containing the particles of pore-forming agent.

With a percentage of pore-forming agent in the polymer blend greater than 50%, the structure of the membranes obtained tends to collapse.

Based on the morphological data obtained, the following optimum values of concentration of the components of the polymer solution were selected:
PEO at 40 wt.%, a concentration giving an ordered structure with regular pore size
vitamin E at 25% (w/w) relative to the initial weight of the PCL/PEO solution.

### 4. Tensile Testing of Porous Membranes of PCL Containing Vitamin E

Using the MTS® QTest™/10 tester, tensile tests were performed on porous membranes of PCL (PCL 80i, PCL 70i, PCL 60i) and on porous membranes of PCL containing vitamin E (PCL 80i+vitE, PCL 70i+vitE, PCL 60i+vitE), to evaluate their mechanical characteristics.

The tensile tests were performed at room temperature, applying the same conditions for all the samples: 50 N load cell and speed of the cross-beam equal to 10 mm/min.

The tensile elastic modulus (E) was calculated, for all the samples prepared, in the linear portion of the stress-strain curve (strain 0-5%).

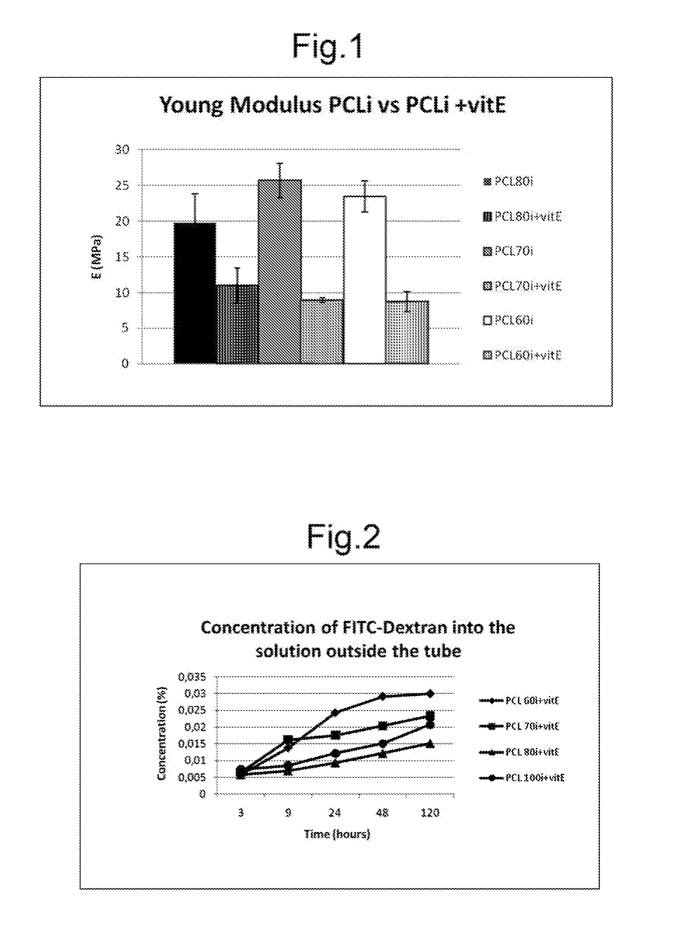

FIG. 1 shows the mean values of Young's modulus of samples PCL 80i, PCL 70i, PCL 60i, PCL 80i+vitE, PCL 70i+vitE, PCL 60i+vitE. The data are presented as mean ± standard deviation.

On comparing the various membranes, it can be seen that the presence of vitamin E has an effect on the mechanical characteristics of the samples analysed. Analysis of the histogram of FIG. 1 in fact shows that the values of Young's modulus are significantly higher for the series of porous membranes of PCL not containing vitamin E than the porous membranes containing the antioxidant.

Based on the data presented relating to mechanical characteristics, it could be concluded that:
the optimum amount of PEO for the production of porous membranes (40 wt.%) does not alter the rigidity and the maximum load of the material, whereas it increases its toughness considerably
the addition of vitamin E leads to plasticization of porous membranes of PCL with reduction of the maximum load and of the values of Young's modulus.

### 5. Evaluation of the Permeability of Porous Membranes of PCL Containing Vitamin E

Permeability tests were carried out on porous membranes based on PCL and containing vitamin E, using a model compound having characteristics of fluorescence: dextran labelled with fluorescein isothiocyanate (FITC-dextran) from SIGMA.

The porous membranes PCL 80i+vitE, PCL 70i+vitE, PCL 60i+vitE, all containing vitamin E at 25% (w/w) relative to the PCL/PEO initial mixture, as indicated in example 2, were rolled up to form small tubes, closed at one end and laterally, to permit the insertion of solutions of FITC-dextran and, finally, closed at the end that was still open. The tubes were closed using a cyanoacrylate adhesive.

The FITC-dextran FD-4, which has molecular weight of 5 kDa and a Stokes radius of 14 Å, was used for these experiments. The FITC-dextran compounds are regarded as model molecules for the permeability tests, as they simulate the dimensions of the nutrients present in the organism (for example glucose, having a Stokes radius of 3.6 Å; sodium chloride, having a Stokes radius of 1.4 Å).

In particular, a solution was prepared at 1% (w/v) of FITC-dextran FD-4 in PBS (3.5 ml), which was poured into a 5 ml test tube and agitated manually. Using a syringe, the tubes were filled with about 160 μl of the solution stated above. The tubes were immersed in 10 ml of PBS, and at prearranged time intervals of 3 hours, 9 hours, 24 hours, 48 hours, and 5 days, all of the release solution was collected, was transferred to new test tubes and was stored in a refrigerator until the moment of evaluation of the release of FITC-dextran from the inside to the outside of the tube. Moreover, at each time interval the release solution collected was replaced with fresh PBS solution.

The amount of FITC-dextran present in PBS corresponds to the total amount of FITC-dextran that had permeated through the porous walls of the tube, since the ends had been sealed with cyanoacrylate. Each release solution has a different concentration of FITC-dextran, in relation to the porosity of the walls (pore density and pore size).

The amount of FITC-dextran FD-4 released was evaluated by measuring the absorbance of the release solutions at prearranged time intervals, using a UV-Vis spectrophotometer. The absorbance of the release solutions was compared with that of the calibration straight line of absorbance of solutions of FITC-dextran FD-4 in PBS as a function of the concentration of FITC-dextran FD-4.

FIG. 2 shows a graph for the release of FITC-dextran from the inside to the outside of the tube. The percentage concentration (mol/l) of FITC-dextran present in the solution outside the tube is shown as a function of time. Curves of the release of FITC-dextran from membranes with different porosities are shown, obtained starting from PCL/PEO blends and containing VIT E (25%).

Based on the data shown, it can be seen that an increase in the concentration of pore-forming agent present in the starting solution corresponds to an increase in the number of FD-4 model molecules that pass through the walls of the tube. Divergent results were recorded for the membrane samples PCL 100i+vitE and PCL 80i+vitE, since the latter have lower permeability relative to the membrane samples of PCL alone. However, statistical analysis did not show significant differences between the two samples examined

In particular, based on statistical analysis, no substantial differences in permeability were found between the tubes produced starting from the mixtures of composition PCL/PEO 100/0, PCL/PEO 80/20 and PCL/PEO 70/30 (+VIT E 25%). In contrast, the permeability behaviour of the tube produced starting from the PCL/PEO 60/40 polymer blend +VIT E 25% was found to be significantly different from all the other samples analysed.

Among the samples analysed, the tube of PCL/PEO 60/40 therefore displays the best characteristics of permeability to the FD-4 particles. Use of this porous membrane would therefore permit the passage of nutrients present in the organism, for example glucose or sodium chloride, which have a Stokes radius less than that of the model molecule.

### 6. Preparation of Coating Membranes of PCL and PLA on Polymer Mesh Supports

Using the solvent casting technique described above, membranes were prepared based on PCL/PEO and PLA/PEO blends with the compositions shown in Table 3 and the latter were used for coating the polypropylene (PP) mesh. Various concentrations of the solutions were assessed, to analyse the relation between solution concentration (and hence presence of polymer) and the thickness of the coating obtained.

For preparing the coating membranes, the system consisting of the polymers and the solvent was stirred by magnetic stirring under a hood to facilitate dissolution. The solvents used for obtaining the mesh coatings were:
acetone (C₃H₆O) (Fluka), at about 40° C. for dissolving PCL;
dichloromethane (CH₂Cl₂) (Fluka) at room temperature for dissolving PLA.

Complete dissolution of the components in acetone and dichloromethane was attained after about 24 hours for the PCL/PEO blends and for PCL; after about 1 hour for PLA; after about 24 hours for the PLA/PEO blends.

The details relating to the coating of PP mesh are presented in Table 3. Table 3 shows the polymers used for coating the mesh (the solute) and the solvent used for dissolving them. It also shows the concentrations of the solutions prepared and the thicknesses both of the mesh provided with coating at two different points (mesh: threads, not threads), and of the coating membrane alone. The last column of the table refers to the weight of the mesh plus the coating, determined before and after extraction of the pore-forming polymer. For the coatings that do not contain the pore-forming agent, the initial and final weights coincide. The symbol “*” indicates the transparency of the coating, which is one of the characteristics of an ideal prosthesis, as it does not create visual difficulties in positioning the latter.

TABLE 3
COATINGS

| | | | Thickness (μm) | | | | |
|---|---|---|---|---|---|---|---|
| | | | | Mesh | | Weight (g) | |
| | | Solution | | | Not | Initial | Final |
| Solute | Solvent (30 ml) | % (w/v) | Membrane | Threads | threads | weight | weight |
| PCL | Acetone | 3 | 90 | 575 | 540 | 0.5463 | |
| PCL | Acetone | 5 | 180 | 510 | 500 | 0.7818 | |
| PCL | Acetone | 8 | 210 | 575 | 500 | 0.9396 | |
| PCL/PEO 80/20 | Acetone | 5 | 170 | 600 | 640 | 0.8486 | 0.7074 |
| PCL/PEO 60/40 | Acetone | 5 | 130 | 550 | 550 | 0.5487 | 0.3694 |
| PLA* | Dichloromethane | 3 | 70 | 600 | 590 | 0.5210 | |
| PLA* | Dichloromethane | 5 | 110 | 600 | 580 | 0.8144 | |
| PLA* | Dichloromethane | 8 | 150 | 590 | 570 | 1.2394 | |
| PLA/PEO 80/20 | Dichloromethane | 5 | 150 | 600 | 580 | 0.6834 | 0.6381 |
| PLA/PEO 60/40 | Dichloromethane | 5 | 120 | 580 | 560 | 0.5945 | 0.5206 |

The polypropylene mesh (area 5 cm²), placed in a glass Petri dish with a diameter of 12 cm (area 113 cm²), was coated with 30 ml of solution (see Table 3) and the solvent was evaporated for 24 hours under a fume hood. Next, the meshes coated with mixtures containing the pore-forming agent were immersed in water in order to obtain porous coatings.

The thickness of the coating and of the system mesh plus coating was measured with a micrometer. Because of the weft of the mesh, the system mesh plus coating is not of uniform thickness, therefore it was necessary to measure the part with denser weft (see Table 3—column “Mesh thickness threads”) and that with less dense weft (see Table 3—column “Mesh thickness not threads”).

The amount and the concentration of the polymers to be used in the solution for making coatings that are homogeneous and not excessively thick, so that they are not inflexible and difficult to handle, were evaluated. For both coatings in PCL and PLA, the best characteristics were obtained when they were made using a solution with polymer concentration of 5% in chloroform. The use of solutions at 3% concentration leads to formation of a layer of polymer on the mesh that is too thin, and can barely be manipulated without damaging it. It also proves rather difficult to keep the system mesh + coating intact when removing it from the Petri dish in which it was prepared. The coating produced starting from a polymer solution at 8% is very rigid, and therefore unsuitable for insertion in the abdominal cavity.

### 7. Morphological Analysis (SEM) of Coating Membranes of PCL and PLA on Polymer Mesh Supports

### PCL

Both faces of the prosthetic device mesh-PCL coating (concentration of the polymer solution for manufacture equal to 5% w/v) were analysed: the lower face, i.e. the face not coated, shows, in the foreground, the PP mesh and the coating on the back of the mesh; the upper face, i.e. the coated face, shows the polymer coating covering the threads of the PP mesh, deposited by the solvent casting technique. The efficiency of the solvent casting technique is demonstrated by the completeness and homogeneity of the coating obtained. These characteristics of the membrane do not change on adding the pore-forming agent and vitamin E to the polymer solution.

After removal of the pore-forming agent by immersion in water, the coating becomes porous and remains well adhered to the PP mesh.

### PLA

The lower surface of the prosthetic device mesh-PLA coating was analysed. It was observed that the PLA coating is much smoother than that of pure PCL. It was also observed that the mesh is well anchored to the polymer coating, since the thread of the mesh is embedded in the membrane, suggesting that the PLA polymer might be an excellent alternative candidate for the manufacture of non-stick coatings.

1-14. (canceled)

15. A method of manufacturing a prosthetic device, having a substantially planar support and a porous coating membrane of polymeric material formed on at least one face of the support, the method comprising:
i) laying on at least one face of the support of the device a solution of the polymeric material dissolved in a first solvent, the solution comprising particles of a pore-forming material dispersed therein;
ii) drying the solution by evaporation of the first solvent, thereby obtaining a membrane of polymeric material formed on at least one face of the support of the device, the membrane comprising particles of a pore-forming material dispersed therein; and
iii) dipping the prosthetic device obtained in step ii) in a second solvent capable of dissolving the particles of the pore-forming material, thereby forming pores in the membrane.

16. The method of claim 15, wherein the polymeric material of the porous coating membrane is selected from the group consisting of: polycaprolactone (PCL), polylactic acid (PLA), polyglycolic acid (PGA), polyurethane (PU) and copolymers thereof

17. The method of claim 15, wherein the pore-forming material is selected from the group consisting of polyethylene oxide (PEO), polyvinylpyrrolidone (PVP), polyvinyl alcohol (PVA), glucose and sodium chloride (NaCl).

18. The method of claim 15, wherein the solution of polymeric material comprises polycaprolactone (PCL) and polyethylene oxide (PEO).

19. The method of claim 18, wherein the proportion of polycaprolactone (PCL) to polyethylene oxide (PEO) is within the range of about 90/10 to about 50/50.

20. The method of claim 19, wherein the proportion of polycaprolactone (PCL) to polyethylene oxide (PEO) is selected from 90/10, 80/20, 70/30, 60/40 or 50/50.

21. The method of claim 15, wherein the solution of polymeric material comprises vitamin E.

22. The method of claim 21, wherein the concentration of vitamin E is between about 5 and about 50% by weight of the total weight of the solution.

23. The method of claim 22, wherein the concentration of vitamin E is about 25% by weight of the total weight of the solution.

24. The method of claim 15, wherein the support is a surgical mesh.

25. The method of claim 24, wherein the surgical mesh comprises polypropylene and/or polyethylene terephthalate.

26. A prosthetic device, comprising a substantially planar support and a porous coating membrane which comprises polymeric material formed on at least one face of said support, the membrane being obtainable by the method of claim 15.

27. The device of claim 26, wherein the support of the device is a surgical mesh.

28. The device of claim 27 wherein the surgical mesh comprises polypropylene and/or polyethylene terephthalate.

29. The device of claim 28, wherein the surgical mesh is a porous or macroporous ultra-light woven polypropylene mesh.

30. The device of claim 29, wherein the surgical mesh is a prosthesis for hernia repair, for the repair and reinforcement of the abdominal wall and the inguinal region of the pelvic floor or for the treatment of urinary incontinence.
